(19) United States
(12) Patent Application Publication
(10) Pub. No.: US 2013/0202544 A1
(43) Pub. Date: Aug. 8, 2013

(54) THICKENED COMPOSITONS OF HIGH PH

(71) Applicants: Linda C. Foltis, Nutley, NJ (US); Anand Ramchandra Mahadeshwar, Pompton Lakes, NJ (US); Jose Martinez-Santiago, New York, NY (US)

(72) Inventors: Linda C. Foltis, Nutley, NJ (US); Anand Ramchandra Mahadeshwar, Pompton Lakes, NJ (US); Jose Martinez-Santiago, New York, NY (US)

(73) Assignee: ISP Investments Inc., Wilmington, DE (US)

(21) Appl. No.: 13/695,141

(22) PCT Filed: Apr. 29, 2011
(86) PCT No.: PCT/US11/34515

§ 371 Date: Jan. 9, 2013

(30) Foreign Application Priority Data
Apr. 30, 2010 (US) 61329772

(51) Int. Cl. A61K 8/81; A61Q 5/04

(52) U.S. Cl. CPC A61K 8/8176; A61Q 5/04
USPC 424/70.4

(57) ABSTRACT

Provided herein are thickened compositions produced upon assembling a first sub-formulation having a hydroxide, and at least one second sub -formulation having an alkaline material, wherein at least one sub-formulation is thickened by lightly-to moderately-crosslinked PVP. Unlike other compositions not having the lightly-to moderately-crosslinked PVP, those described herein maintain or even build viscosity when the first and second sub -formulations are blended. The compositions may be utilized in the formulation of personal care and performance chemicals, such as hair relaxers, hair straighteners, depilators, degreasers, paint strippers, and cleaners. Methods for enhancing product handling and performance are also described.

### FIELD OF THE INVENTION

Disclosed are personal care and performance chemicals systems and compositions that comprise at least a first sub-formulation having a hydroxide and at least one second sub-formulation having an alkaline material, wherein at least one sub-formulation is thickened by lightly-to moderately-crosslinked PVP. In one embodiment the thickened composition is assembled from two or more parts and is a hair relaxer, a hair straightener, or a depilatory.

### DESCRIPTION OF RELATED ART

The styling of hair requires a skillful blend of science and art to attain that perfect look. Among the many hair styling techniques that are available today, hair relaxing is a fundamental method, which can be used to partially or completely straighten curly or wavy hair, or used to produce uniform hair before proceeding onto other styling methods (like perming, waving, curling, coloring, or cutting).

Hair relaxing, also known as lanthionization, originally developed from soaps containing excessive quantities of lye, and the caustic treatment often irritated the scalp, hands, and eyes. Today, hair relaxers and straighteners have been formulated to improve both the user experience and the hair style itself. These advances allow hair relaxing/straightening to be practiced using techniques at home using over-the-counter products, and in professional salons.

In order to “relax” or “straighten” hair from curls, waves, and/or cowlicks (whorls) it is necessary to chemically alter hair's protein and keratin structure, or by weakening cystine bonds so that the hair fiber extends into a more linear fashion. To achieve this result, hair can be treated with a high pH formula, assembled from one- or two-subformulations that are blended immediately prior to use to generate the high pH active, Although the one-sub-formulation approach offers convenience, degradation of the active ingredient occurs over time, even as it sits on store shelves, which reduces the effectiveness of such one sub-formulation hair relaxers and straighteners. For many consumers, there is a performance preference for the two sub-formulation products, wherein the active ingredient (and high pH) are produced by mixing the two sub-formulations immediately before use.

Yet, these two sub-formulation hair relaxers and straighteners display some unusual characteristics that are linked to the in situ reaction that creates the necessary high pH active ingredient. Most typically, these products are sold as a thick crème relaxer (the first part) along with an activator (the second part) that often has the consistency resembling water. However, the final, blended product does not possess the thick, rich characteristics of the crème relaxer, but, due to a drop in viscosity, is notably much thinner. It is important to note that this decrease cannot be attributed by virtue to the pH of the resulting product. The crème relaxer (first part) itself is extremely basic, and a pH in excess of 11 is common (see Comparative Examples 1-4). Instead, this drop in viscosity is due to the in situ reaction itself. Because of this thinning behavior, the crème relaxer sometimes is formulated with additional thickener in order to create a final, blended product that is sufficiently thick. Despite high levels of thickener, the blended, final product usually has less than half of the crème relaxer's viscosity. However, the hair relaxer or hair straightener still may be susceptible to deficiencies like difficult handling/blending, poor coverage, or unsatisfactory user safety (e.g., runs into the eyes, or drips onto clothes or the floor).

Hence, needed are new compositions for one and two sub-formulation hair relaxers and straighteners, particularly those that do not exhibit the viscosity drop when blended together. Also needed are uses of these improved products to improve product handling and performance attributes.

Compositions disclosed herein contain lightly-to moderately-crosslinked poly(N-vinyl-2-pyrrolidone). This polymer was first introduced in U.S. Pat. No. 5,073,614. In that patent it is taught to be the precipitation polymerization product of N-vinyl-2-pyrrolidone monomer in an organic solvent, such as an aliphatic hydrocarbon solvent (particularly cyclohexane or heptane) or an aromatic hydrocarbon (such as toluene) in the presence of about 0.2% to 1% by weight of a crosslinking agent. The fine, white powders thus produced have an aqueous gel volume from about 15 mL to about 150 mL per gram of polymer, and a Brookfield viscosity in 5% aqueous solution of at least about 10,000 cP.

This lightly-to moderately-crosslinked poly(N-vinyl-2-pyrrolidone) (PVP) polymer also was the subject of U.S. Pat. No. 5,139,770. It provides examples wherein this polymer is incorporated into different types of personal care compositions.

U.S. Pat. No. 5,716,634 discloses a lightly-crosslinked N-vinyl lactam polymer in form of stable, clear, flowable, homogenized hydrogel, which may be used as a carrier for cosmetic/pharma actives for hair or skin use. A controlled release drug-delivery composition comprising a lightly-crosslinked poly(N-vinyl-2-pyrrolidone) polymer is the subject of U.S. Pat. No. 5,252,611. Also, the production of lightly-crosslinked poly(N-vinyl-2-pyrrolidone)polymer in an oil-in-water or water-in-oil emulsion is described in U.S. Pat. No. 6,177,068.

A summary of some properties of light-to moderately-crosslinked PVP is given in Shih, J. S., “Characteristics of lightly crosslinked poly(N-vinylpyrrolidone),” Polymer Materials: Science & Engineering Preprint, 72, 374, 1995.

Still more information on this lightly crosslinked PVP polymer is given in the following U.S. Pat. Nos. 5,162,417; 5,242,985; 5,268,117; 5,312,619; 5,470,884; 5,534,265; 5,614,583; 5,618,522; 5,622,168; 5,564,385; 5,645,859; 5,658,577; 5,663,258; 5,759,524; 5,843,881; 5,919,440; 5,968,528; 5,973,359; 5,997,887; 5,997,890; 6,001,377; 6,024,942; 6,174,533; 6,582,711; and 7,390,478. Related disclosure also is provided in U.S. patent applications 2003/0215413; 2007/0122501; and 2007/0154435. Also related are U.S. Statutory Registrations USH 2,013 and 2,043. Also related are German patents DE 69,533,239; 69,813,874; 69,814,066; 69,816,439; 69,818,037; 69,831,326; and 69,906,265. Related disclosure also is provided in European patent specification EP 777,465; and in PCT applications WO 1999/052501; 1999/052502; 2000/101523; 2000/048555; 2000/048568 and 2000/048569.

All of the above patents, patent applications, and Statutory Registrations, and the mentioned Shih article above are hereby incorporated in their entirety by reference.

It is desired to resolve the observations noted with commercial hair relaxers, as well as other personal care and performance chemicals compositions.

It also is desired to improve the user experience with these compositions, such that product handling, blending, coverage, spreadability, and user safety are enhanced.

Also, it is desired to provide methods improving the aesthetic hair styling qualities achieved by the hair relaxer (or hair straightener) formulas, such as improved hair shine, uniformity, manageability, and alignment.

### SUMMARY OF THE INVENTION

It has been discovered that lightly-to moderately-crosslinked PVP effectively thickens compositions having an in situ reaction between a first sub-formulation comprising a hydroxide and at least a second sub-formulation comprising an alkaline material.

In one aspect, the invention provides effective particle stabilization with or without the need to create an emulsion or microemulsion. The stabilized particulate suspension can be a hydroxide-containing part, an alkaline-containing part, or both. Upon blending these two sub-formulations, which result in the in situ reaction forming the high pH active, a thickened system is created that cannot be made without the lightly-to moderately-crosslinked PVP. Yet, two sub-formulations are not required in order to stabilize the particle dispersion. It was found that particle dispersions of one sub-formulation systems also are stabilized using effective amounts of lightly-to moderately-crosslinked PVP. Alternatively, more than two sub-formulations also can be effectively thickened.

In another aspect, the invention provides compositions of exceptional thickness after the above-described first and second sub-formulations are mixed. The resulting viscosity is more than expected when compared to similar formulas that do not contain the lightly-to moderately-crosslinked PVP.

In yet another aspect, the invention provides for compositions that build viscosity when the first and second sub-formulations are blended together. This product performance is unlike related products that create the high pH active from an in situ reaction.

In accordance with another aspect, the present application relates to a system comprising (A) a first sub-formulation comprising a hydroxide; and (B) a second sub-formulation comprising an alkaline material, wherein at least one of (A) and (B) comprises a lightly-to moderately-crosslinked poly(N-vinyl-2-pyrrolidone).

One skilled in the art will recognize the invention is primarily directed to personal care and performance chemicals applications. Non-limiting examples of such compositions are two sub-formulation hair relaxers, hair straighteners, and depilatory products, as well as degreasers, drain openers, paint strippers, cleaners, leather depilatory, or leather tanning compositions.

Related to this thickening ability, the invention provides methods for improving the performance attributes of the described compositions. These improved performance attributes include improved product handling, consistency, application, spreadability and coverage, as well as enhanced user safety.

Additionally, the invention provides for methods of providing better looking hair with regard to shine, manageability, and uniformity of appearance that is achieved through the use of a two sub-formulation thickened composition that involves the in situ reaction of a hydroxide and an alkaline material.

### BRIEF DESCRIPTION OF THE DRAWINGS

FIG. 1 is a graph of Brookfield viscosity as a function of percent addition of lightly-to moderately-crosslinked PVP addition to Sub-formulation A (no oil phase), as described in accordance with Example 2.

FIG. 2 is a graph of pH as a function of percent addition of lightly-to moderately-crosslinked PVP addition to Sub-formulation A, as described in accordance with Example 3.

FIG. 3 is a graph of Brookfield viscosity as a function of percent addition of lightly-to moderately-crosslinked PVP addition to Sub-formulation A, as described in accordance with Example 5.

FIG. 4 is a graph of pH as a function of percent addition of lightly-to moderately-crosslinked PVP addition to Sub-formulation A, as described in accordance with Example 6.

FIG. 5 is a graph of Brookfield viscosity as a function of percent addition of lightly-to moderately-crosslinked PVP as described in accordance with Example 10.

FIG. 6 is a graph of pH as a function of percent addition of lightly-to moderately-crosslinked as described in accordance with Example 10.

FIG. 7 is a graph of Brookfield viscosity as a function of stability storage time and storage condition for liquid activators (Sub-formulation B) containing 4% lightly-to moderately-crosslinked PVP, as described in accordance with Example 11.

FIG. 8 is a graph of pH as a function of stability storage time and storage condition for liquid activators (Sub-formulation B) containing 4% lightly-to moderately-crosslinked PVP, as described in accordance with Example 11.

FIG. 9 is a graph of Brookfield viscosity as a function of stability storage time and storage condition for liquid activators (Sub-formulation B) containing 5% lightly-to moderately-crosslinked PVP, as described in accordance with Example 11.

FIG. 10 is a graph of pH as a function of stability storage time and storage condition for liquid activators (Sub-formulation B) containing 5% lightly-to moderately-crosslinked PVP, as described in accordance with Example 11.

FIG. 11 is a graph of Brookfield viscosity as a function of stability storage time and storage condition for liquid activators (Sub-formulation B) containing 6% lightly-to moderately-crosslinked PVP, as described in accordance with Example 11.

FIG. 12 is a graph of pH as a function of stability storage time and storage condition for liquid activators (Sub-formulation B) containing 6% lightly-to moderately-crosslinked PVP, as described in accordance with Example 11.

### DETAILED DESCRIPTION

It has been discovered that lightly-to moderately-crosslinked PVP effectively thickens compositions of at least two sub-formulations and high pH that create an in situ reaction. The first sub-formulation is water- or water and oil-based having a hydroxide, while the at least second sub-formulation also is water- or water and oil-based having one or more alkaline materials. The lightly-to moderately-crosslinked PVP may be added to either the first, second, or both first and second sub-formulations to thicken the composition.

Distinctly different from other thickeners and contrary to expectation, lightly-to moderately-crosslinked PVP was found to be an exemplary thickener. The blended products exhibit higher viscosity compared to existing products, and, in particular embodiments, actually build a viscosity that is greater than either of the two sub-formulations.

It also was discovered that in certain cases lightly-to moderately-crosslinked PVP stabilizes particle dispersions without forming an emulsion or microemulsion. In this manner, some embodiments include thickened compositions having at least the two described sub-formulations but without an oil-phase. Analogous compositions made without the lightly-to moderately-crosslinked PVP are unacceptable due to phase separation.

Due to the inherent complexity in these compositions, their ingredients, product forms, and uses, it will be appreciated that definitions of terms will help describe various embodiments of the invention.

The term personal care composition (or formulation) refers to compositions intended for topical use on a mammal, including, man, horses, cats, and dogs. These compositions include skin, hair, scalp, foot, or lip compositions, including those compositions that can be purchased with and without a doctor's prescription. Non-limited examples of personal care compositions include those to straighten, relax, or remove hair. The personal care compositions also may comprise other active and non-active ingredients to assist in delivery, spreadability, emolliency, film formation, stability, and/or thickening.

The term performance chemicals composition (or formulation) refers to non-personal care compositions that serve a broad variety of applications, and include non-limiting compositions such as: adhesives; agricultural, biocides, coatings, electronics, household-industrial-institutional (HI&I), inks, membranes, metal fluids, oilfield, paper, paints, plastics, printing, plasters, and wood-care compositions.

The term sub formulation refers to one or more compositions, each comprising one or more ingredient(s), that are assembled to yield a formulation. The simplest example is a single sub-formulation having a single ingredient that itself represents a formulation. More complex formulations can be devised from multiple sub-formulations, each of which comprise various ingredient(s). Hair relaxers that are produced by blending two or more sub-formulations are just a few examples of sub-formulations.

The term lightly-to moderately-crosslinked PVP, unless otherwise noted, specifically refers to polymer essentially consisting of lightly-to moderately-crosslinked poly(N-vinyl-2-pyrrolidone) having at least one of the following characteristics: (1) an aqueous swelling parameter defined by its gel volume from about 15 mL/g to about 300 mL/g, more preferably from about 15 mL/g to about 250 mL/g, and most preferably from about 15 mL/g to about 150 mL/g, or (2) a Brookfield viscosity of 5% lightly-to moderately-crosslinked PVP in water at 25° C. of at least 2,000 cP, more preferably of at least about 3,000 cP, and most preferably of at least about 10,000 cP. Disclosure for these parameter ranges is provided in U.S. Pat. No. 5,073,614 and in Shih, J. S., et al. (1995). Synthesis methods for the lightly-to moderately-crosslinked PVP are disclosed in a number of references, including U.S. Pat. Nos. 5,073,614; 5,654,385; and 6,177,068. It is appreciated by a polymer scientist skilled in the art that the method of synthesis is immaterial, inasmuch as the produced polymer achieves at least one of the above-defined parameters.

For example, U.S. patent '614 discloses different crosslinkers and crosslinker amounts that yield lightly-to moderately-crosslinked PVP suitable for the present invention. The effect of crosslinker amount on swell volume and viscosity is graphically presented in Shih, J. S., et al. (1995). Thus, the lightly-to moderately-crosslinked PVP may be produced by the precipitation polymerization method of the '614 patent, by the hydrogel method described in the '385 patent, or by the non-aqueous, heterogeneous polymerization method of the '068 patent. Certainly, other techniques are contemplated to synthesize this polymer, provided the product meets the aqueous swelling parameter and Brookfield viscosity requirements.

Final product viscosities may slightly vary for compositions containing lightly-to moderately-crosslinked PVP made by these different methods. Nonetheless, these variations are within the scope of the invention, as the lightly-to moderately-crosslinked PVPs thicken low pH compositions.

Unless otherwise specified, “lightly-to moderately-crosslinked PVP” does not refer to swellable but water-insoluble crosslinked PVP, such as the type sold into commercial trade under the trade name Polyclar® by International Specialty Products, which differs from the above-described lightly-to moderately-crosslinked PVP.

The term viscosity refers to the proportionality coefficient between shear stress and shear rate, and describes a composition's resistance to flow. Because viscosity is dependent on shear rate, specific measurement information (such as viscometer, flow apparatus/spindle, and shear rate) is required to properly define viscosity. As used herein, viscosity refers to the proportionality coefficient determined from low shear rate, rotational flow, especially the viscosity measured by the Brookfield LVT and Brookfield RVT viscometers typically operating at 10 revolutions per minute (rpm) at 25° C., although other temperatures are specified in the present invention. References describing the Brookfield measurement of viscosities include the following, each of which is hereby incorporated in its entirety by reference: Thibodeau, L., “Measuring viscosity of pastes,” American Laboratory News, June 2004; McGregor, R. G., “Shelf life: does viscosity matter?” Pharmaceutical Online, Oct. 31, 2007; and McGregor, R. G., “When ointments disappoint, the viscosity story,” Brookfield Engineering brochure.

The term high pH refers to a pH greater than 11.

### First Embodiment: Thickened Compositions of at Least Two Sub-Formulations

As mentioned briefly earlier, it has been discovered that lightly-to moderately-crosslinked PVP uniquely thickens compositions of at least two sub-formulations that produce an in situ reaction between a hydroxide in the first sub-formulation and an alkaline material in the at least second part.

These two sub-formulations may comprise, or be essentially free or even completely free of an oil-phase, meaning they are essentially water-based only. It was discovered that the addition of lightly-to moderately-crosslinked PVP stabilizes particle dispersions that otherwise settle and cause phase separation after blending.

An example of this aspect of the invention is illustrated in Example 1 (below), wherein a water-based crème relaxer (first sub-formulation) was prepared with varying levels of lightly-to moderately-crosslinked PVP in water. Without this polymer, the crème relaxer was unstable and the calcium hydroxide dispersion settled. If this unstable crème relaxer is used, then the final, blended product exhibits phase separation due to this inhomogeneity and, thus, is unsuitable for use. However, by adding 2% (based on the weight of the crème relaxer) or more of this thickening polymer, stable water-based crème relaxers were produced.

In this example, the addition level of the lightly-to moderately-crosslinked PVP may be selected to provide a high viscosity of this crème relaxer, since it will be blended with a second sub-formulation that has the viscosity of water (around 1 cP). For example, the crème relaxer may comprise from about 0.5% (w/w) to about 10% (w/w), more particularly from about 1.5% to 8%, and even more particularly from about 2% to 5% lightly-to moderately-crosslinked PVP. The spirit of the invention is maintained even at higher addition levels of lightly-to moderately-cmsslinked PVP, but may not be favored if excessively high viscosity inhibits blending, mixing, or preparing the first sub-formulation or the final blended product. Lower addition levels also are contemplated.

By comparative example, a measurable and sustained drop in viscosity results in commercially available hair relaxers (or straighteners) having more than one sub-formulation which are blended together. The final product viscosity of these commercial formulas was significantly lower than that of the crème relaxer, and the final product viscosities did not exceed 42,000 cP.

This first sub-formulation comprises one or more hydroxides, such as an alkali or alkaline earth metal hydroxide (e.g., calcium hydroxide, potassium hydroxide, lithium hydroxide, or sodium hydroxide), or ammonium hydroxide. The addition level of this hydroxide in the first sub-formulation is not limited, inasmuch as it provides an effective final product for the intended use. For example, hair relaxers and straighteners produced from two or more sub-formulations of the invention were prepared containing 5% (w/w) (with regard to the total mass of the first sub-formulation) of the hydroxide.

In addition to the first part, the second sub-formulation comprises an alkaline material for the in situ reaction with the hydroxide of the first sub-formulation when the two sub-formulations are blended. This alkaline material may be in the form of particles, especially fine, micronized, or even nano-sized particles, dispersed in the second part, or may be in the form of an emulsion or solution. In one embodiment this alkaline material is a carbonate, such as lithium carbonate, sodium carbonate, potassium carbonate, or guanidine carbonate, Blends of these alkaline materials may be used. Alternatively, the alkaline material may be a glycolate or a thioglycolate. Non-limiting examples of glycolates include: ammonium glycolate and diammonium dithiodiglycolate; and non-limiting examples of thioglycolates include: ammonium thioglycolate, butyl thioglycolate, calcium thioglycolate, 2-methoxyethyl thioglycolate, 2-ethoxyethyl thioglycolate, 2-ethoxypropyl thioglycolate, ethanolamine thioglycolate, ethyl thioglycolate, glyceryl thioglycolate, isooctyl thioglycolate, isopropyl thioglycolate, magnesium thioglycolate, methyl thioglycolate, potassium thioglycolate, sodium thioglycolate, and strontium thioglycolate. Of course, combinations of these materials may be used.

In one aspect of this embodiment, the compositions provided herein may be personal care formulas, such as two sub-formulation hair relaxers and straighteners, or depilatories. The lightly-to moderately-crosslinked PVP may be present in either the first or second sub-formulation because the final product is effectively thickened.

Like the first part, this second sub-formulation also may include optional ingredients, especially those known to one skilled in the related fields of hair relaxers and straighteners, and chemical depilators (e.g., thioglycolic acid, ammonium thioglycolate), and the aforementioned performance chemicals formulations. The amount of this alkaline material is not particularly limited inasmuch as the quantity is sufficient to lead to the in situ reaction when blended with the first part, and result in a utile product. In one non-limiting aspect, approximately equal molar ratios (plus or minus 20%) may be used of the hydroxide of the first sub-formulation and the alkaline material of the second sub-formulation. Ratios outside this range may also work but would result in an excess of one component.

Upon mixing the first and second sub-formulations, the resulting reaction produces a high pH product (typically, but not necessarily always, with a pH in excess of 11) that allows it to serve in the described end uses, especially as a hair relaxer, hair straightener, or depilator. For example, this blended product may have a viscosity of at least 500 cP, as they are sufficiently viscous to avoid being watery and runny. In different aspects, the viscosity may be more than about 1,000 cP, or more than about 3,000 cP.

As mentioned, the particle dispersions that are stabilized by effective amounts of lightly-to moderately-crosslinked PVP may be those of two sub-formulations. This polymer also thickens one sub-formulation systems having at least one particle dispersion, as well as those formulations assembled from more than two sub-formulations (for example, a hair relaxer of three or four sub-formulations).

Alternatives to this first example described earlier also are embraced by the invention. For example, the first sub-formulation described above also may contain one or more oil-based phases, meaning these phases are not water-soluble. There are many advantages to incorporating such oil-based phases into the first sub-formulation. They may impart conditioning, moisturizing, protecting, texture/feel, detangling, and/or shine agents, especially for products that contact the scalp, skin, or hair. Especially preferred are oil-based phases with an affinity for the scalp in order to protect it from the high pH of the hair relaxer/straightener. Or, such oil-based phases may assist in solubilizing and delivering hydrophobic co-ingredients which may be difficult or impossible to accomplish in a water-only based first sub-formulation. Examples of suitable oil-based phases include hydrocarbon oils and non-hydrocarbon oils. Blends of these oils may be used. A description of suitable oils will be provided after first describing the viscosities of these thickened compositions having one or more oil-based phases.

The incorporation of an oil-based phase enables a broader range of viscosities than capable in the water-phase only approach. Without being bound by theory, it is thought that the lightly-to moderately-crosslinked PVP helps to disperse/reduce droplet size of the oil-based phase, especially when presented as an emulsion or micro-emulsion. By this approach viscosities of 70,000 cP or more can be attained in the blended, final product with even small addition levels of the lightly-to moderately-crosslinked PVP, e.g., 1% (w/w) to 2% (w/w). Depending on the formulation strategy, the viscosity of the final, blended product can be lower, equal to, or even higher than that of the crème relaxer.

When the hydroxide first sub-formulation contains both water- and oil-based sub-formulations and lightly-to moderately-crosslinked PVP, then blending it with the alkaline second sub-formulation may result in product viscosities less than the first part. Despite the drop in viscosity, it is surprising that product viscosities of 67,000 cP are generated with as little as 1% lightly-to moderately-crosslinked PVP addition. These viscosities are considerably higher than those of many commercial products.

In another scenario, the first sub-formulation again contains the water- and oil-based sub-formulations, and the second sub-formulation contains the lightly-to moderately-crosslinked PVP. Once blended together, three viscosity regions can be generated. At low lightly-to moderately-crosslinked PVP addition, the final, blended product viscosity may be less than that of the crème relaxer. Unexpectedly, it is possible for the product viscosity actually to exceed that of the first sub-formulation at higher levels of the thickening polymer to the second sub-formulation. This result is essentially without counterpart for these in situ reactions creating a high pH active. At intermediate lightly-to moderately-crosslinked PVP addition levels, the product viscosity is about equivalent to the first sub-formulation (crème relaxer).

Thus, a smaller amount of lightly-to moderately-crosslinked PVP is needed to reach a target viscosity when the first sub-formulation has water- and oil-based phases compared to formulas having only a water-phase.

Examples of oils include, but are not restricted to, those that find use in personal care compositions. Among these are petrolatum and mineral oil (i.e., paraffinic oils, naphthenic oils, and aromatic oils). Also suitable are the different vegetable oils (e.g., coconut, corn, cottonseed, olive, palm, peanut, rapeseed, Canola, safflower, sesame, soybean, sunflower, almond, cashew, hazelnut, macadamia, mongongo, pecan, pine nut, evening primrose, blackcurrant see, borage seed, and grape seed), Also known are the essential oils from the berries, seeds, bark, wood, rhizome, leaves, resin, flowers, peel, or roots of plants (e.g., allspice, juniper, almond, anise, celery, cumin, nutmeg, cassia, cinnamon, sassafras, camphor, cedar, rosewood, sandalwood, agar wood, galangal, ginger, basil, bay leaf, common sage, eucalyptus, lemon grass, melaleuca, oregano, patchouli, peppermint, pine, rosemary, spearmint, tea tree, thyme, wintergreen, chamomile, clary sage, clove, geranium, hops, hyssop, jasmine, lavender, manuka, marjoram, orange, rose, ylang-ylang, bergamot, grapefruit, lemon, tangerine, and valerian). Essential oils may be employed for integrating an enhanced olfactory and/or tactile experience into the hair relaxing process.

Other oils also are known to those skilled in the art, and may be used with the invention. One class is the family of silicone oils, being oils based at least in part on silicon-oxygen linkages, and may be branched or unbranched. For example, silicone oils are those used in personal care formulations, where they may serve as conditioning agent.

The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and mixtures thereof.

Suitable polyalkyl siloxanes include polydimethyl siloxanes with terminal trimethyl silyl groups or terminal dimethyl silanol groups (dimethiconol) and polyalkyl (C1-C20) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes preferably having a number-average molecular weight between 200,000 and 1,000,000, used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers. In one embodiment the organo-modified silicone is an aminofunctional silicone. Broadly speaking, these polymers contain at least one amine group and at least one silicon atom. These polymers represent a broad array of chemistries that may be ideal for creating the disclosed ultraviolet-absorbing compounds. For example, aminoalkylsiloxanes and aminoalkoxysiloxanes are but two examples of this polymer family, which can be further reacted to yield chemistries that include polyimides, polyureas, and polyurethanes.

Examples of aminofunctional silicones include isostearamidopropyl dimethylamine gluconate (and) propylene glycol amine-functional silicones such as those offered for commercial sale by The Lubrizol Corporation (Wickliffe, Ohio). Also available are a number of aminopropyl-terminated polydimethylsiloxanes, N-ethylamino-isobutyl terminated-polydimethyl siloxanes, aminopropylmethylsiloxane-dimethylsiloxane copolymers, aminoethyl-aminopropyl-methylsiloxane-d methyls oxane copolymers, aminoethyl-aminoisobutyl-methylsiloxane-dimethylsiloxane copolymers, and aminoethyl-aminopropylmethoxysiloxane-dimethylsiloxane copolymers, all of which are offered for commercial sale by Gelest, Inc. (Morrisville, Pa.). Blends of polymers having amine units also are contemplated.

The silicones may be used in the form of emulsions, nano-emulsions, or micro-emulsions.

When oil-based phases are included in the present compositions, it may be desirable to include one or more emulsifiers (including those that produce microemulsions) with the oil-based phase in the first part. These emulsifiers help to stabilize the multi-phase composition to avoid separation, changes in viscosity and/or pH over time, and may assist in delivering the active ingredient(s).

Not only may the lightly-to moderately-crosslinked PVP be formulated in the first sub-formulation, as in the above-described two examples, but this polymer also may be included in the second sub-formulation. The amount of lightly-to moderately-crosslinked PVP in the second sub-formulation may be selected to produce suitable viscosities of the second sub-formulation and/or final blended product. For example, as little as 0.5% (w/w) or 1% (w/w) of this polymer in the second sub-formulation can create blended product viscosities of about 15,000 cP −20,000 cP, values that resemble viscosities of conventional two sub-formulation hair relaxers and straighteners. More generally, the second sub-formulation may comprise from about 0.5% (w/w) to about 10% (w/w), more particularly from about 1% to 7%, and even more particularly from about 2% to 5% lightly-to moderately-crosslinked PVP.

A surprising and unexpected increase in viscosity was measured when lightly-to moderately-crosslinked PVP was added to the second sub-formulation at addition levels up to 3% (wlw). Essentially no increase in viscosity was measured for the second sub-formulation, but an increase of about 20,000 cP was recorded for the final, blended product. The addition of 2% (w/w) of the PVP to the second sub-formulation can create a final, blended product that equals the original viscosity of the crème relaxer (first part). Again, even higher addition levels are embraced by the invention inasmuch as the compositions are effectively thickened following the in situ reaction. The choice is left to one skilled in the art such that appropriate and effective viscosities of the second sub-formulation and final blended product result.

It is understood that the invention is not limited to hair relaxers, hair straighteners, and depilatories assembled from two sub-formulations, or even just to personal care formulations. The ability of lightly-to moderately-crosslinked PVP to thicken these high pH, formulations extends to other compositions such as performance chemicals compositions. Contemplated is the use of the invention to produce degreasers, drain openers, paint strippers, cleaners, and leather depilator, leather tanning formulations, and other known high pH performance chemicals compositions to name a few.

Acknowledging the many ways personal care and performance chemicals compositions may be used, it is within the scope of the invention that the thickened compositions may have the form of a solution, a cream, an ointment, a lotion, an oil-in-water emulsion, a water-in-oil emulsion, a shampoo, a spray, or a gel.

### Optional: Additional Formulation Ingredients and Adjuvants

Due to the requirements of end performance, it is expected that the compositions of this invention will be used together with other additives to further enhance the properties of the finished product. Such ingredients may be incorporated without altering the scope of the current invention, and may be included in order to produce the necessary products.

Among these optional formulary ingredients are conditioning agents, especially when used for hair. The conditioning agent can be a protein or hydrolyzed cationic or non-cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C1-C18 alkyl.

Hydrolyzed proteins include Croquat L, in which the quaternary ammonium groups include a C12 alkyl group, Croquat M, in which the quaternary ammonium groups include C10-C18 alkyl groups, Croquat S in which the quaternary ammonium groups include a C18 alkyl group and Crotein Q in which the quaternary ammonium groups include at least one C1-C18 alkyl group. These products are sold by Croda.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein, 2-N-stearoyl amino-octadecane-1,3-diol, 2-N-behenoyl amino-octadecane-1,3-diol, 2-N-[2-hydroxy-palmitoyl]-amino-oetadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl)inalonamide, N-(2-hydroxy ethyl)-N-(3-cetoxyl-2-hydroxy propyl) amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide. Suitable examples include mono-, di-, or tri-alkyl quaternary ammonium compounds with a counterion such as a chloride, methosulfate, tosylate, etc. including, but not limited to, cetrimonium chloride, dicetyldimonium chloride, behentrimonium methosulfate, and the like. The presence of a quaternary ammonium compound in conjunction with the polymer described above reduces static and enhances combing of hair in the dry state. The polymer also enhances the deposition of the quaternary ammonium compound onto the hair substrate thus enhancing the conditioning effect of hair.

The conditioning agent can be any fatty amine known to be useful as a conditioning agent; e.g. dodecyl, cetyl or stearyl amities, such as stearamidopropyl dimethylamine.

The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic ester acids including mono-, di-, tri- and tetra- carboxylic acids.

The conditioning agent can be a fluorinated or perfluorinated oil. The fluoridated oils may also be fluorocarbons such as fluoramines, e.g., perfluorotributylamine, fluoridated hydrocarbons, such as perfluorodecahydronaphthalene, fluoroesters, and fluoroethers.

Of course, mixtures of two or more conditioning agents can be used.

The conditioning agent or agents can be present in an amount of 0.001% to 20%, preferably from 0.01% to 10%, and even more preferably from 0.1% to 3% by weight based on the total weight of the final composition.

Optionally, compositions of the invention may contain hair brightening agents, such as those described in U.S. Pat. No. 6,007,585, which is hereby incorporated in its entirety by reference. Other hair brightening, bleaching, or coloring agents also may be used. They help to remove discolorants from the hair, impart a brighter and/or lighter hair color, or even completely change the shade, color intensity, or color itself. Preferred are oxidizing agents such as the alkali metal salts of chromate, chlorate and the like, as well as reducing salts of alkali metal sulfite, bisulfite, hydrosulfite, and related compounds, as well as blue or violet coloring agents used for hair treatment.

The composition of the invention can contain one or more protecting agents to prevent or limit the degrading effects of natural physical and/or chemical assaults on the keratinous materials.

The antioxidants or antiradical agents can be selected from phenols such as BHA (tert-butyl-4-hydroxyanisole), BHT (2,6-di-tert-butyl-p-cresol), TBHQ (tert-butyl hydroquinone), polyphenols such as proanthocyanodic oligomers, flavonoids, hindered amines such as tetra amino piperidine, erythorbic acid, polyamines such as spermine, cysteine, glutathione, superoxide dismutase, and lactoferrin.

The vitamins can be selected from ascorbic acid (vitamin C), vitamin E, vitamin E acetate, vitamin E phosphate, B vitamins such as B3 and B5, vitamin PP, vitamin A, and derivatives thereof. The provitamins can be selected from panthenol and retinol.

The protecting agent can be present in an amount 0.001% to 20% by weight, preferably from 0.01% to 10% by weight, and more preferably 0.1 to 5% by weight of the total weight of the final composition.

In addition, the compositions according to the invention advantageously include at least one surfactant, which can be present in an amount of 0.1% and 60% preferably 1% and 40%, and more preferably 5% and 30% by weight based on the total weight of the composition. The surfactant may be chosen from among anionic, amphoteric, or non-ionic surfactants, or mixtures of them known to be useful in personal care compositions.

Additional thickeners or viscosity increasing agents may be included in the composition of the invention, such as: acetamide MEA; acrylamide/ethalkonium chloride acrylate copolymer; acrylamide/ethyltrimonium chloride acrylate/ethalkonium chloride acrylate copolymer; acrylamides copolymer; acrylamide/sodium acrylate copolymer; acrylamide/sodium acryloyldimethyltaurate copolymer; acrylates/acetoacetoxyethyl methacrylate copolymer; acrylates/beheneth-25 methacrylate copolymer; acrylates/C10-C30 alkyl acrylate crosspolymer; acrylates/ceteth-20 itaconate copolymer; acrylates/ceteth-20 methacrylate copolymer; acrylates/laureth-25 methacrylate copolymer; acrylates/palmeth-25 acrylate copolymer; acrylates/palmeth-25 itaconate copolymer; acrylates/steareth-50 acrylate copolymer; acrylates/steareth-20 itaconate copolymer; acrylates/steareth-20 methacrylate copolymer; acrylates/stearyl methacrylate copolymer; acrylates/vinyl isodecanoate crosspolymer; acrylic acidlacrylonitrogens copolymer; adipic acid/methyl DEA crosspolymer; agar; agarose; alcaligenes polysaccharides; algin; alginic acid; almondamide DEA; almondamidopropyl betaine; aluminum/magnesium hydroxide stearate; ammonium acrylates/acrylonitrogens copolymer; ammonium acrylates copolymer; ammonium acryloyldimethyltaurate/vinyl formamide copolymer; ammonium acryloyldimethyltaurate/VP copolymer; ammonium alginate; ammonium chloride; ammonium polyacryloyldimethyl taurate; ammonium sulfate; amylopectin; apricotamide DEA; apricotamidopropyl betaine; arachidyl alcohol; arachidyl glycol; arachis hypogaea (peanut) flour; ascorbyl methylsilanol pectinate; astragalus gummifer gum; attapulgite; avena sativa (oat) kernel flour; avocadamide DEA; avocadarnidopropyl betaine; azelamide MEA; babassuamide DEA; babassuamide MEA; babassuamidopropyl betaine; behenamide DEA; behenamide MEA; behenamidopropyl betaine; behenyl betaine; bentonite; butoxy chitosan; caesalpinia spinosa gum; calcium alginate; calcium carboxymethyl cellulose; calcium carrageenan; calcium chloride; calcium potassium carbomer; calcium starch octenylsuccinate; C20-40 alkyl stearate; canolamidopropyl betaine; capramide DEA; capryl/capramidopropyl betaine; carbomer; carboxybutyl chitosan; carboxymethyl cellulose acetate butyrate; carboxymethyl chitin; carboxymethyl chitosan; carboxymethyl dextran; carboxymethyl hydroxyethylcellulose; carboxymethyl hydroxypropyl guar; carnitine; cellulose acetate propionate carboxylate; cellulose gum; ceratonia siliqua gum; cetearyl alcohol; cetyl alcohol; cetyl babassuate; cetyl betaine; cetyl glycol; cetyl hydroxyethylcellulose; chimyl alcohol; cholesterol/HDI/pullulan copolymer; cholesteryl hexyl dicarbamate pullulan; citrus aurantium dulcis (orange) peel extract; cocamide DEA; cocamide MEA; cocamide MIPA; cocamidoethyl betaine; cocamidopropyl betaine; cocamidopropyl hydroxysultaine; coco-betaine; coca-hydroxysultaine; coconut alcohol; coco/oleatnidopropyl betaine; coco-Sultaine; cocoyl sarcosinamide DEA; cornamide/cocamide DEA; cornamide DEA; croscarmellose; crosslinked bacillus/glucose/sodium glutamate ferment; cyamopsis tetragonoloba (guar) gum; decyl alcohol; decyl betaine; dehydroxanthan gum; dextrin; dibenzylidene sorbitol; diethanolaminooleamide DEA; diglycol/CHDM/isophthalates/SIP copolymer; dihydroabietyl behenate; dihydrogenated tallow benzylmonium hectorite; dihydroxyaluminum aminoacetate; dimethicone/PEG-10 crosspolymer; dimethicone/PEG-15 crosspolymer; dimethicone propyl PG-betaine; dimethylacrylamide/acrylic acid/polystyrene ethyl methacrylate copolymer; dimethylacrylamide/sodium acryloyldimethyltaurate crosspolymer; disteareth-100 IPDI; DMAPA acrylates/acrylic acidlacrylonitrogens copolymer; erucamidopropyl hydroxysultaine; ethylene/sodium acrylate copolymer; gelatin; gellan gum; glyceryl alginate; glycine soja (soybean) flour; guar hydroxypropyltrimonium chloride; hectorite; hyaluronic acid; hydrated silica; hydrogenated potato starch; hydrogenated tallow; hydrogenated tallowamide DEA; hydrogenated tallow betaine; hydroxybutyl methylcellulose; hydroxyethyl acrylate/sodium acryloyldimethyl taurate copolymer; hydroxyethylcellulose; hydroxyethyl chitosan; hydroxyethyl ethylcellulose; hydroxyethyl stearamide-MIPA; hydroxylauryl/hydroxymyristyl betaine; hydroxypropylcellulose; hydroxypropyl chitosan; hydroxypropyl ethylenediamine carbomer; hydroxypropyl guar; hydroxypropyl methylcellulose; hydroxypropyl methylcellulose stearoxy ether; hydroxypropyl starch; hydroxypropyl starch phosphate; hydroxypropyl xanthan gum; hydroxystearamide MEA; isobutylene/sodium maleate copolymer; isostearamide DEA; isostearamide MEA; isostearamide mIPA; isostearamidopropyl betaine; lactamide MEA; lanolinamide DEA; lauramide DEA; lauramide MEA; lauramide MIPA; lauramide/myristamide DEA; lauramidopropyl betaine; lauramidopropyl hydroxysultaine; laurimino bispropanediol; lauryl alcohol; lauryl betaine; lauryl hydroxysultaine; lauryl/myristyl glycol hydroxypropyl ether; lauryl sultaine; lecithinamide DEA; linoleamide DEA; linoleamide MEA; linoleamide MIPA; lithium magnesium silicate; lithium magnesium sodium silicate; macrocystis pyrifera (kelp); magnesium alginate; magnesium/aluminum/hydroxide/carbonate; magnesium aluminum silicate; magnesium silicate; magnesium trisilicate; methoxy PEG-22/dodecyl glycol copolymer; methylcellulose; methyl ethylcellulose; methyl hydroxyethylcellulose; microcrystalline cellulose; milkamidopropyl betaine; minkamide DEA; minkamidopropyl betaine; MIPA-myristate; montmorillonite; Moroccan lava clay; myristamide DEA; myristamide MEA; myristamide MIPA; myristamidopropyl betaine; myristamidopropyl hydroxysultaine; myristyl alcohol; myristyl betaine; natto gum; nonoxynyl hydroxyethylcellulose; oatamide MEA; oatamidopropyl betaine; octacosanyl glycol isostearate; octadecene/MA copolymer; oleamide DEA; oleamide MEA; oleamide MIPA; oleamidopropyl betaine; oleamidopropyl hydroxysultaine; oleyl betaine; olivamide DEA; olivamidopropyl betaine; oliveamide MEA; palmamide DEA; palmamide MEA; palmamide MIPA; palmamidopropyl betaine; palmitamide DEA; palmitamide MEA; palmitamidopropyl betaine; palm kernel alcohol; palm kernelamide DEA; palm kernelamide MEA; palm kernelamide MIPA; palm kernelamidopropyl betaine; peanutamide MEA; peanutamide MIPA; pectin; PEG-800; PEG-crosspolymer; PEG-150/decyl alcohol/SMDI copolymer; PEG-175 diisostearate; PEG-190 distearate; PEG-15 glyceryl tristearate; PEG-140 glyceryl tristearate; PEG-240/HDI copolymer bis-decyltetradeceth-20 ether; PEG-100/IPDI copolymer; PEG-180/laureth-50/™MG copolymer; PEG-10/lauryl dimethicone crosspolymer; PEG-15/lauryl dimethicone crosspolymer; PEG-2M; PEG-5M; PEG-7M; PEG-9M; PEG-14M; PEG-20M; PEG-23M; PEG-25M; PEG-45M; PEG-65M; PEG-90M; PEG-115M; PEG-160M; PEG-180M; PEG-120 methyl glucose trioleate; PEG-180/octoxynol-40/™MG copolymer; PEG-150 pentaerythrityl tetrastearate; PEG-4 rapeseedamide; PEG-150/stearyl alcohol/SMDI copolymer; phaseolus angularis seed powder; polianthes tuberosa extract; polyacrylate-3; polyacrylic acid; polycyclopentadiene; polyether-1; polyethylene/isopropyl maleate/MA copolyol; polyglyceryl-3 disiloxane dimethicone; polyglyceryl-3 polydimethylsiloxyethyl dimethicone; polymethacrylic acid; polyquaternium-52; polyvinyl alcohol; potassium alginate; potassium aluminum polyacrylate; potassium carbomer; potassium carrageenan; potassium chloride; potassium palmate; potassium polyacrylate; potassium sulfate; potato starch modified; PPG-2 cocamide; PPG-1 hydroxyethyl caprylamide; PPG-2 hydroxyethyl cocamide; PPG-2 hydroxyethyl coco/isostearamide; PPG-3 hydroxyethyl soyamide; PPG-14 laureth-60 hexyl dicarbamate; PPG-14 laureth-60 isophoryl dicarbamate; PPG-14 palmeth-60 hexyl dicarbamate; propylene glycol alginate; PVP/decene copolymer; PVP montmorillonite; pyrus cydonia seed; pyrus malus (apple) fiber; rhizobian gum; ricebranamide DEA; ricinoleamide DEA; ricinoleamide MEA; ricinoleamide MIPA; ricinoleamidopropyl betaine; ricinoleic acid/adipic acid/AEEA copolymer; rosa multiflora flower wax; sclerotium gum; sesamide DEA; sesamidopropyl betaine; sodium acrylate/acryloyldimethyl taurate copolymer; sodium acrylates/acrolein copolymer; sodium acrylates/acrylonitrogens copolymer; sodium acrylates copolymer; sodium acrylates crosspolymer; sodium acrylate/sodium acrylamidomethylpropane sulfonate copolymer; sodium acrylates/vinyl isodecanoate crosspolymer; sodium acrylate/vinyl alcohol copolymer; sodium carbomer; sodium carboxymethyl chitin; sodium carboxymethyl dextran; sodium carboxymethyl beta-glucan; sodium carboxymethyl starch; sodium carrageenan; sodium cellulose sulfate; sodium chloride; sodium cyclodextrin sulfate; sodium hydroxypropyl starch phosphate; sodium isooctylene/MA copolymer; sodium magnesium fluorosilicate; sodium oleate; sodium palmitate; sodium palm kernelate; sodium polyacrylate; sodium polyacrylate starch; sodium polyacryloyldimethyl taurate; sodium polygamma-glutamate; sodium polymethacrylate; sodium polystyrene sulfonate; sodium silicoaluminate; sodium starch octenylsuccinate; sodium stearate; sodium stearoxy PG-hydroxyethylcellulose sulfonate; sodium styrene/acrylates copolymer; sodium sulfate; sodium tallowate; sodium tauride acrylates/acrylic acid/acrylonitrogens copolymer; sodium tocopheryl phosphate; solanum tuberosum (potato) starch; soyamide DEA; soyamidopropyl betaine; starch/acrylates/acrylamide copolymer; starch hydroxypropyltrimonium chloride; stearamide AMP; stearamide DEA; stearamide DEA-distearate; stearamide DIBA-stearate; stearamide MEA; stearamide MEA-stearate; stearamide MIPA; stearamidopropyl betaine; steareth-60 cetyl ether; steareth-100/PEG-136/HDI copolymer; stearyl alcohol; stearyl betaine; sterculia urens gum; synthetic fluorphlogopite; tallamide DEA; tallow alcohol; tallowamide DEA; tallowamide MEA; tallowamidopropyl betaine; tallowamidopropyl hydroxysultaine; tallowamine oxide; tallow betaine; tallow dihydroxyethyl betaine; tamarindus indica seed gum; tapioca starch; TEA-alginate; TEA-carbomer; TEA-hydrochloride; trideceth-2 carboxamide MEA; tridecyl alcohol; triethylene glycol dibenzoate; trimethyl pentanol hydroxyethyl ether; triticum vulgare (wheat) germ powder; triticum vulgare (wheat) kernel flour; triticum vulgare (wheat) starch; tromethamine acrylates/acrylonitrogens copolymer; tromethamine magnesium aluminum silicate; undecyl alcohol; undecylenamide DEA; undecylenamide MEA; undecylenamidopropyl betaine; welan gum; wheat germamide DEA; wheat germamidopropyl betaine; xanthan gum; yeast beta-glucan; yeast polysaccharides and zea mays (corn) starch.

Preferred thickeners or viscosity increasing agents include carbomer, aculyn and Stabileze®, e.g. crosslinked acrylic acid, crosslinked poly(methylvinyl ether/maleic anhydride) copolymer, acrylamides, carboxymethyl cellulose and the like.

These formulations typically have a liquid or liquid-like carrier that aids to distribute, disperse, and/or dissolve the formulation ingredients, including the lightly-to moderately-crosslinked PVP. Selection of these carriers is not limited, and examples of liquid carriers include water, alcohols, oils, esters, and blends thereof.

The compositions described herein also can contain one or more additional additives chosen from conditioning agents, protecting agents, such as, for example, hydrosoluble, antiradical agents, antioxidants, vitamins, ultraviolet absorbers, and pro-vitamins, fixing agents, oxidizing agents, reducing agents, dyes, cleansing agents, anionic, cationic, nonionic and amphoteric surfactants, thickeners, perfumes, pearlizing agents, stabilizers, pH adjusters, filters, preservatives, cationic and nonionic polyether associative polyurethanes, polymers other than the cationic polymer described herein, vegetable oils, mineral oils, synthetic oils, polyols such as glycols and glycerol, silicones, aliphatic alcohols, colorants, bleaching agents, highlighting agents and sequestrants. These additives are present in the composition according to the invention in proportions that may range from 0% to 20% by weight in relation to the total weight of the composition. The precise amount of each additive may be easily determined by an expert in the field according to its nature and its function.

If it is desired that the final product protects the hair from ultraviolet radiation, it may be desirable to include one or more UV absorbers. In this context, the terms ultraviolet and UV mean electromagnetic radiation, especially solar electromagnetic radiation, with a wavelength from about 100 nm to about 400 nm, and includes the UV-A, UV-B, and UV-C subclassifications of such radiation. The term UV-A means ultraviolet electromagnetic radiation with a wavelength from about 320 nm to about 400 nm, and includes UV-Al (from about 340 nm to about 400 nm) and UV-A2 (from about 320 nm to about 340 nm). The term UV-B means ultraviolet electromagnetic radiation with a wavelength from about 290 nm to about 320 mn. The term UV-C means ultraviolet electromagnetic radiation with a wavelength from about 200 nm to about 290 nm. Finally, the term UV absorber means any entity that absorbs, scatters, and/or reflects any wavelength of UV radiation.

Suitable UV absorbers that may be included most likely will depend on local regulations. Because the rules governing the names and usage levels evolve over time, it is impossible to include every UV absorber that may be used with the invention. Typical UV absorbers include, without limitation: octyl salicylate; pentyl dimethyl PABA; octyl dimethyl PABA; benzophenone-1; benzophenone-6; 2-(2H-benzotriazole-2-yl)-4,6-di-tert-pentylphenol; ethyl-2-cyano-3,3-diphenylacrylate; homomenthyl salicylate; bis-ethylhexyloxyphenol methoxyphenyl triazine; methyl-(1,2,2,6,6-pentamethyl-4-piperidyl)-sebacate; 2-(2H-benzotriazole-2-yl)-4-methylphenol; diethylhexyl butamido triazone; amyl dimethyl PABA; 4,6-bis(octylthiomethyl)-o-cresol; CAS number 65447-77-0; red petroleum; ethylhexyl triazone; octocrylene; isoamyl-p-methoxycinnamate; drometrizole; titanium dioxide; 2,4-di-tert-butyl-6-(5-chloro-2H-benzotriazole-2-yl)-phenol; 2-hydroxy-4-octyloxybenzophenone; benzophenone-2; diisopropyl methylcinnamate; PEG-25 PABA; 2-(1,1-dimethylethyl)-6-[[3-(1,1-demethylethyl)-14[3-(1,1-2-hydroxy-5-methylphenyl]methyl-4-methylphenyl acrylate; drometrizole trisiloxane; menthyl anthranilate; butyl methoxydibenzoylmethane; 2-ethoxyethyl p-methoxycinnatnate; benzylidene camphor sulfonic acid; dimethoxyphenyl-[1-(3,4)]-4,4-dimethyl 1,3-pentanedione; zinc oxide; N,N′-hexane-1,6-diylbis[3-(3,5-di-tert-butyl-4-hydroxyphenylpropionamide)]; pentaerythritol tetrakis[3-(3 ,5-di-tert-butyl-4-hydroxyphenyl)propionate]; 2,6-di-tert-butyl-4-[4,6-bis(octylthio)-1,3,5-triazin-2-ylamino]phenol; 2-(2H-benzotriazole-2-yl)-4,6-bis(1-methyl-1-phenylethyl)phenol; trolamine salicylate; diethylanolamine p-methoxycinnamate; polysilicone-15; CAS number 152261-33-1; 4-methylbenzylidene camphor; bisoctrizole; N-phenyl-benzenamine; reaction products with 2,4,4-trimethylpentene; sulisobenzone; (2-ethylhexyl)-2-cyano-3,3-diphenylacrylate; digalloyl trioleate; polyacrylamido methylbenzylidene camphor; glyceryl ethylhexanoate dimethoxycinnamate; 1,3-bis-[(2′-cyano-3′,3′-diphenylacryloyl)oxy]-2,2-bis-{[(2′-cyano-bis-(2,2,6,6-tetramethyl-4-piperidyl)-sebacate; benzophenone-5; 1,3,5-tris(3,5-di-tert-butyl-4-hydroxybenzyl)-1,3,5-triazine-2,4,6(1H,3H,5H)-trione; hexamethylendiamine; benzophenone-8; ethyl-4-bis(hydroxypropyl)aminobenzoate; 6-tert-butyl-2-(5-chloro-2H-benzotriazole-2-yl)-4-methylphenol; p-aminobenzoic acid; 3,3′,3″,5,5′,5″-hexa-tert-butyl-α-α′-α″-(mesitylene-2,4,6-triyl)tri-p-cresol; lawsone with dihydroxyacetone; benzophenone-9; benzophenone-4; ethylhexyl dimethoxy benzylidene dioxoimidazoline propionate; N,N′-bisformyl-N,N′-bis-(2,2,6,6-tetramethyl-4-piperidinyl)-; 3-benzylidene camphor; terephthalylidene dicamphor sulfonic acid; camphor benzalkonium methosulfate; bisdisulizole disodium; etocrylene; ferulic acid; 2-(2H-benzotriazole-2-yl)-4-(1,1,3,3-tetramethylbutyl)-phenol; 4,6-bis(dodecylthiomethyl)-o-cresol; β-2-glucopyranoxy propyl hydroxy benzophenone; phenylbenzimidazole sulfonic acid; benzophenone-3; diethylamine hydroxybenzoyl hexylbenzoate; 3′,3′-diphenylacryloyl)oxy]methyl)-propane; ethylhexyl p-methoxycinnamate, and blends thereof.

Any known conditioning agent is useful in the personal care compositions of this invention. Conditioning agents function to improve the cosmetic properties of the hair, particularly softness, thickening, untangling, feel, and static electricity and may be in liquid, semi-solid, or solid form such as oils, waxes, or gums. Similarly, any known skin altering agent is useful in the compositions of this invention. Preferred conditioning agents include cationic polymers, cationic surfactants and cationic silicones.

Conditioning agents may be chosen from synthesis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds.

The synthesis oils include polyolefins, e.g., poly-a-olefins such as polybutenes, polyisobutenes and polydecenes. The polyolefins can be hydrogenated.

The mineral oils suitable for use in the compositions of the invention include hexadecane and oil of paraffin.

A list of suitable animal and vegetable oils comprises sunflower, corn, soy, avocado, jojoba, squash, raisin seed, sesame seed, walnut oils, fish oils, glycerol tricaprocaprylate, Purcellin oil or liquid jojoba, and blends thereof.

Suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot.

Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax, and blends thereof

The cationic polymers that may be used as a conditioning agent include those known to improve the cosmetic properties of hair treated by detergent compositions. The expression “cationic polymer” as used herein, indicates any polymer containing cationic groups and/or ionizable groups in cationic groups. The cationic polymers used generally have a molecular weight the average number of which falls between about 500 Da and 5,000,000 Da and preferably between 1000 Da and 3,000,000 Da.

The preferred cationic polymers are chosen from among those containing units including primary, secondary, tertiary, and/or quaternary amine groups that may either form part of the main polymer chain or a side chain.

Useful cationic polymers include known polyamine, polyaminoamide, and quaternary polyammonium types of polymers, such as:

(1) homopolymers and copolymers derived from acrylic or methacrylic esters or amides. The copolymers can contain one or more units derived from acrylamides, methacrylamides, diacetone acrylamides, acrylamides and methaerylamides, acrylic or methacrylic acids or their esters, vinyllactams such as vinyl pyrrolidone or vinyl caprolactam, and vinyl esters. Specific examples include: copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyl oxyethyl trimethyl ammonium methosulfate; copolymers of vinyl pyrrolidone/dialkylaminoalkyl acrylate or methacrylate, optionally quaternized, such as the products sold under the name Gafquat® by International Specialty Products; the dimethyl amino ethyl methacrylate/vinyl caprolactam/vinyl pyrrolidone terpolymers, such as the product sold under the name Gaffix® VC 713 by International Specialty Products; the vinyl pyrrolidone/methacrylamidopropyl dimethylamine copolymer, marketed under the name Styleze® CC 10 by International Specialty Products; and the vinyl pyrrolidone/quaternized dimethyl amino propyl methacrylamide copolymers such as the product sold under the name Gafquat® HS 100 by International Specialty Products (Wayne, N.J.).

(2) derivatives of cellulose ethers containing quaternary ammonium groups, such as hydroxy ethyl cellulose quaternary ammonium that has reacted with an epoxide substituted by a trimethyl ammonium group.

(3) derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl- cellulose grafted with a salt of methacryloyl ethyl trimethyl ammonium, methacrylamidopropyl trimethyl ammonium, or dimethyl diallyl ammonium.

(4) cationic polysaccharides such as those described in U.S. Pat. Nos. 3,589,578 and 4,031,307, guar gums containing cationic trialkyl ammonium groups and guar gums modified by a salt, e.g., chloride of 2,3-epoxy propyl trimethyl ammonium.

(5) polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers.

(6) water-soluble polyamino amides prepared by polycondensation of an acid compound with a polyamine. These polyamino amides may be reticulated.

(7) derivatives of polyamino amides resulting from the condensation of polyalcoylene polyamines with polycarboxylic acids followed by alcoylation by bi-functional agents.

(8) polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347.

(9) the cyclopolymers of alkyl dialyl amine or dialkyl diallyl ammonium such as the homopolymer of dimethyl diallyl ammonium chloride and copolymers of diallyl dimethyl ammonium chloride and acrylamide.

(10) quaternary diammonium polymers such as hexadimethrine chloride.

(11) quaternary polyammonium polymers, including, for example, Mirapol® A 15, Mirapol® AD1, Mirapol® AZ1, and Mirapol® 175 products sold by Miranol

(12) the quaternary polymers of vinyl pyrrolidone and vinyl imidazole such as the products sold under the names Luviquat® FC 905, FC 550, and FC 370 by BASF Corporation.

(13) quaternary polyamines.

(14) reticulated polymers known in the art.

Other cationic polymers that may be used within the context of the invention are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyurethanes, and derivatives of chitin.

Preferred cationic polymers are derivatives of quaternary cellulose ethers, the homopolymers and copolymers of dimethyl diallyl ammonium chloride, quaternary polymers of vinyl pyrrolidone and vinyl imidazole, and mixtures thereof.

The conditioning agent can be any silicone known by those skilled in the art to be useful as a conditioning agent. The silicones suitable for use according to the invention include polyorganosiloxanes that are insoluble in the composition. The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and mixtures thereof.

Suitable polyalkyl siloxanes include polydimethyl siloxanes with terminal trimethyl silyl groups or terminal dimethyl silanol groups (dimethiconol) and polyalkyl (C1-C20) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes preferably having a number-average molecular weight between 200,000 Da and 1,000,000, Da used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers.

The silicones may be used in the form of emulsions, nano-emulsions, or micro-emulsions.

### Second Embodiment of the Invention: Uses of the Thickened Compositions

Not only does the invention provide for thickened compositions, it also describes the uses thereof.

In one aspect of this second embodiment, the thickened compositions are personal care compositions of at least two sub-formulations. Given the high pH these compositions attain, they find special use as hair relaxers, hair straighteners, and depilators. Each category will be described briefly to better define how the invention applies.

The hair relaxers and straighteners of the present application include a number of related compositions that find service in styling hair. As used herein, the term hair refers to the mostly proteinaceous growth from follicles of mammals, including humans. Many different areas of the body grow hair, and of greatest interest is hair of sufficient length that is can be styled, such as hair on top of the head and facial hair. The term hair relaxer describes the general category of related compositions that can find use in chemically softening, swelling, and/or altering the cortical layer and/or weakening cystine bonds to help reduce or eliminate hair curl, wave, and cowlicks (hair whorls). Hair relaxers and straighteners can be used to treat all of the hair, or be applied in an specific areas to avoid overtreatment and the possibility of damaging hear. Thus, in one sense, compositions of the invention can be used to fully or partially relax hair, or fully or partially straighten hair. In practice, additional uses of these compositions are known and included in this second embodiment.

For example, hair relaxers/straighteners also find application in reducing hair fizz and/or reducing hair bulk, as in individuals having coarse or high hair density. Hair relaxers also have utilization in the penning of hair, where the relaxer may be used in a preliminary step to promote uniformity to the hair before perming (e.g., when hair is wavy or curly in one section but straight in another).

Within all of these contexts, the hair relaxer also can be used to make hair softer, easier to comb, detangle hair, and make hair easier to style.

The thickened compositions also find use as depilators, meaning they chemically help to remove hair. Depilators may assume the form of creams, lotions, gels, ointments, and even sprays, depending on formulary aspects and customer preferences. These thickened, at least two-sub-formulation depilators may be used to remove hair from anywhere on the body, like the legs, arms, face, neck, bikini/swimsuit lines, and back. Depilators also are used in the tanning of leather, where these compositions also find application. Being assembled from at least two sub-formulations, the depilators may be dispensed from two, or more preferably a single container designed to blend (or facilitate blending of) the components on application.

As described in the section for optional ingredients, it may be beneficial to formulate the hair relaxers and depilators with conditioners, moisturizers, and/or protectants for skin or hair in order to promote mildness and make the compositions less irritating.

### Third Embodiment of the Invention: Improved Product Handling

After discovering the surprising way that viscosity is maintained or even increased in these high pH compositions wherein the active is produced from an in situ blending reaction, it was then learned that these formulas also improve product handling. Consider the traditional line-up of products having a significant (and undesirable) drop in viscosity when the two (or more) sub-formulations are blended together: The loss in viscosity yields an end-use product that can be runny, thin, and therefore difficult to handle, evenly distribute, spread, or cover the intended application area (e.g., a head of hair).

These product limitations may be lessened or even eliminated so that the product exhibits easier handling and consistency. The maintained or increased viscosities render hair relaxers/straighteners and depilators that are easier to blend, spread, and cover hair. User safety also is improved, since the thicker, high pH compositions are less likely to run into the eyes or drip.

### Fourth Embodiment of the Invention: Enhanced End Results

It is important to recognize that hair relaxing/straightening and styling (or removing hair) concerns more than just the compositions involved. The products are used to enhance physical appearance and achieve an aesthetically appealing attractiveness. To this aspect, the invention also provides a fourth embodiment of enhanced end results. Due to the improved handling, product consistency, spreadability, and coverage properties, compositions of the invention promote styled hair of enhanced shine, manageability, and uniformity. Consequently, hair can be cut and styled better, giving it better alignment, and in the end, presenting a better appearance compared to conventional products that become runny when the two (or more) sub-formulations are mixed.

The following examples are presented to illustrate specific embodiments of the present compositions and methods. These examples should not be interpreted as limitations upon the scope of the invention.

### EXAMPLES

### Comparative Examples 1-4

### Commercial Hair Relaxer Formulas

Four commercial, two sub-formulation hair relaxer products were obtained that listed calcium hydroxide in Sub-formulation A (crème relaxer) and guanidine carbonate in Sub-formulation B (liquid activator) (Table 1). The Brookfield viscosities (η) and pH were measured of each component and the final product blend immediately upon preparation. All measurements were obtained at room temperature (about 22° C.) using a Brookfield RV viscometer with spindle 4-7 (as appropriate).

Extremely high pH (>13) was measured for each final product blend (Sub-formulations A+B), due to the in situ formation of guanidine hydroxide. As a result of this reaction, a 39%-73% drop in viscosity (Δη) was measured for the four products (relative to the viscosity of Sub-formulation A, the crème relaxer) (Table 1).

TABLE 1
Viscosities and pH of four commercial hair relaxers.
sub-formulations

| | | A: crème relaxer | | B: liquid activator | | A + B | | |
|---|---|---|---|---|---|---|---|---|
| manufacturer | brand | pH | η (cP) | pH | η (cP) | pH | η (cP) | Δη* |
| Beautiful | Botanicals | 12.5 | 69,500 | 11.3 | ~1 | 13.0 | 33,600 | −51% |
| Silk Elements | MegaSilk | 12.6 | 120,000 | 10.9 | ~1 | 13.1 | 32,300 | −73% |
| Silk Elements | Sensitive | 12.1 | 90,000 | 10.6 | ~1 | 13.3 | 42,000 | −53% |
| Root Simulator Organic | Olive Oil | 12.3 | 43,000 | 10.7 | ~1 | 13.3 | 26,000 | −39% |

*Relative to the viscosity of the crème relaxer.

### Comparative Example 5

### Hair relaxer Formulation without Lightly-to Moderately-Crosslinked PVP

A hair relaxer formulation of two sub-formulations was prepared (Table 2). Sub-formulation A was a crème relaxer containing calcium hydroxide. Sub-formulation B contained the activator, guanidine carbonate, and water,

The viscosities of A, B, and the final product blend (sub-formulations A+B) were measured at ambient temperature (about 22° C.) using a Brookfield RV viscometer fitted with spindle RV-5. Sub-formulation A, a non-running cream, had a viscosity of 28,000 cP, and sub-formulation B had the viscosity of water (about 1 cP). A substantial and undesirable drop in viscosity was measured when A and B were mixed together in a 3.28 to 1 ratio, respectively. The viscosity of the final product blend was 11,280 cP, corresponding to a 60% drop in viscosity compared to A (Table 3).

TABLE 2
Control hair relaxer formulation of Comparative Example 5.
Sub-formulation A: Créme Relaxer

| | |
|---|---|
| ingredient | mass percent |

Phase 1

| | |
|---|---|
| petrolatum | 16 |
| mineral oil | 16 |
| cetearyl alcohol | 6 |
| polysorbate-60 | 2 |
| PEG-75 lanolin | 2 |

Phase 2

| | |
|---|---|
| deionized water | 51 |
| propylene glycol | 2 |
| calcium hydroxide | 5 |
| total | 100 |

Sub-formulation B: Activator

| ingredient | mass percent |
|---|---|
| water | 75 |
| guanidine carbonate | 25 |
| total | 100 |

TABLE 3
Viscosities of the hair relaxer of Comparative Example 5

| composition | η (cP) | Δη* |
|---|---|---|
| sub-formulation A | 28,000 | |
| sub-formulation B | ~1 | |
| sub-formulations A + B | 11,280 | −60% |

*Relative to the viscosity of sub-formulation A, the créme relaxer.

### Example 1

### Hair Relaxer Formulations with Lightly-Crosslinked PVP in a Water-Based crème Relaxer (Sub-Formulation A)

Six hair relaxer formulations of the invention were prepared in two sub-formulations that yielded guanidine hydroxide when mixed (Table 4). Sub-formulation A was a water-based, one sub-formulation cream relaxer that had varying levels of lightly-to moderately-crosslinked PVP supplied from a 10% solution in water. Sub-formulation B contained the activator, guanidine carbonate. A control also was made, having sub-formulation A of 93% water, 2% propylene glycol, 5% calcium hydroxide, and the sub-formulation B. The ratio of sub-formulation A to sub-formulation B in the blended product was 3.28:1, which provides stoichiometrically equal amounts of calcium hydroxide and guanidine carbonate. One skilled in the art can devise other ratios that are about stoichiometrically equal and still obtain a hair relaxing benefit. For example, the sub-formulations may be blended at ratios of about 0.8-1.2:1 (A to B).

The control hair relaxer formula (without lightly-to moderately crosslinked PVP) was unstable and phase separated.

Quite unexpectedly, stable hair relaxers of the invention were produced with the addition of 2% or more lightly-to moderately-crosslinked PVP to sub-formulation A.

TABLE 4

| Hair relaxer formulations of Example 1 | | | | | | |
|---|---|---|---|---|---|---|
| sub-formulation A: Crème Relaxer | | | | | | |
| ingredient | mass percent | | | | | |
| deionized water | 83 | 73 | 63 | 53 | 43 | 33 |
| propylene glycol | 2 | 2 | 2 | 2 | 2 | 2 |
| calcium hydroxide | 5 | 5 | 5 | 5 | 5 | 5 |
| lightly- to moderately | 10 | 20 | 30 | 40 | 50 | 60 |
| crosslinked PVP, | | | | | | |
| 10% solution | | | | | | |
| total | 100 | 100 | 100 | 100 | 100 | 100 |

sub-formulation B: Activator

| ingredient | mass percent | | | | | |
|---|---|---|---|---|---|---|
| water | 75 | 75 | 75 | 75 | 75 | 75 |
| guanidine carbonate | 25 | 25 | 25 | 25 | 25 | 25 |
| total | 100 | 100 | 100 | 100 | 100 | 100 |

### Example 2

### Viscosities of Hair Relaxers of Example 1

Viscosities were measured for sub-formulation A, sub-formulation B, and the blended, final products of Example 1. A Brookfield RV viscometer fitted with spindle 4-7 (as appropriate) was employed for the viscosity measurements.

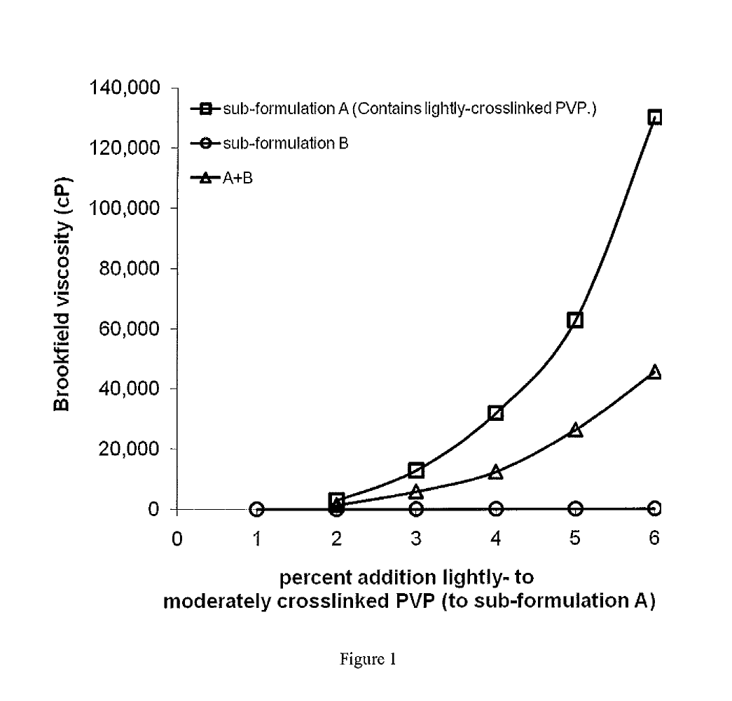

The hair relaxer formulations of Example 1 exhibited remarkably higher viscosities (FIG. 1) than the control. Aesthetically favorable formulations were provided when using from about 3% to about 5% lightly-to moderately-crosslinked PVP.

### Example 3

### pH of Hair Relaxers of Example 1

The pH was measured for sub-formulation A, sub-formulation B, and the combined mixtures of Example 1.

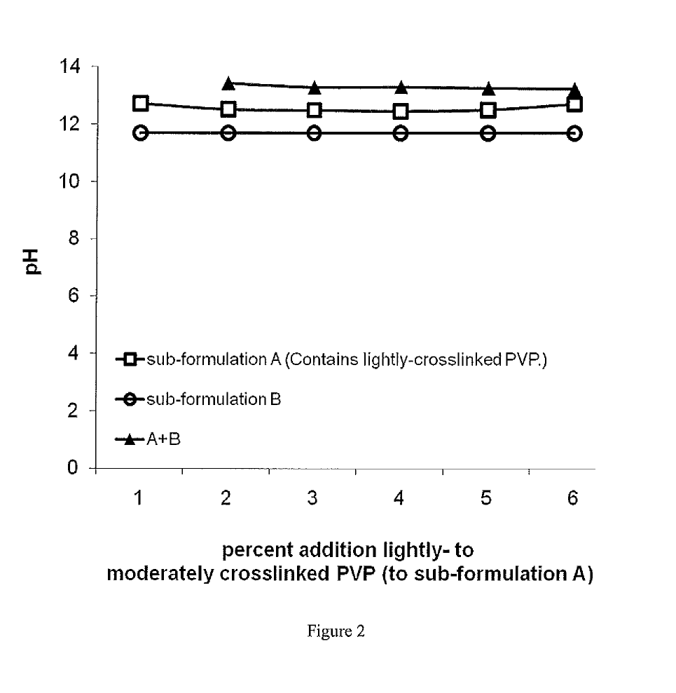

The increase in viscosity reported in Example 1 was not due to a drop in mixture pH, as all components, including the mixture, had a pH greater than 11.75 (FIG. 2).

### Example 4

### Hair Relaxer Formulations with Lightly-to Moderately-Rrosslinked PVP in a Two-Lhase crème Relaxer (Sub-Formulation A).

Six hair relaxer formulations of the invention were prepared in two sub-formulations that yielded guanidine hydroxide when mixed (Table 5). Sub-formulation A was a cream relaxer and contained an oil-based phase 1, and a water-based phase 2 that had varying levels of lightly-to moderately-crosslinked PVP supplied from a 10% solution in water (except for the final two formulation, in which the crosslinked PVP was supplied from a 20% solution in water). Sub-formulation B contained the activator, guanidine carbonate. A control also was made, having sub-formulation A of 51% water, 2% propylene glycol, 5% calcium hydroxide, and the same Sub-formulation B.

TABLE 5
Hair relaxer formulations of Example 4
Sub-formulation A: Crème Relaxer

| | Phase 1 | | | | | |
|---|---|---|---|---|---|---|
| ingredient | mass percent | | | | | |
| petrolatum | 16 | 16 | 16 | 16 | 16 | 16 |
| mineral oil | 16 | 16 | 16 | 16 | 16 | 16 |
| cetearyl alcohol | 6 | 6 | 6 | 6 | 6 | 6 |
| polysorbate-60 | 2 | 2 | 2 | 2 | 2 | 2 |
| PEG-75 lanolin | 2 | 2 | 2 | 2 | 2 | 2 |
| | Phase 2 | | | | | |
| ingredient | mass percent | | | | | |
| deionized water | 41 | 31 | 21 | 11 | 1 | 21 |
| propylene glycol | 2 | 2 | 2 | 2 | 2 | 2 |
| calcium hydroxide | 5 | 5 | 5 | 5 | 5 | 5 |
| lightly- to moderately | 10 | 20 | 30 | 40 | 50 | 30^{†} |
| crosslinked PVP, | | | | | | |
| 10% solution | | | | | | |
| total | 100 | 100 | 100 | 100 | 100 | 100 |

Sub-formulation B: Activator

| ingredient | mass percent | | | | | |
|---|---|---|---|---|---|---|
| water | 75 | 75 | 75 | 75 | 75 | 75 |
| guanidine carbonate | 25 | 25 | 25 | 25 | 25 | 25 |
| total | 100 | 100 | 100 | 100 | 100 | 100 |

^{†}20% solution

### Example 5

### Viscosities of Hair Relaxers of Example 4

Viscosities were measured for sub-formulation A, sub-formulation B, and the blended, final products of Example 4. A Brookfield RV viscometer was employed for the viscosity measurements.

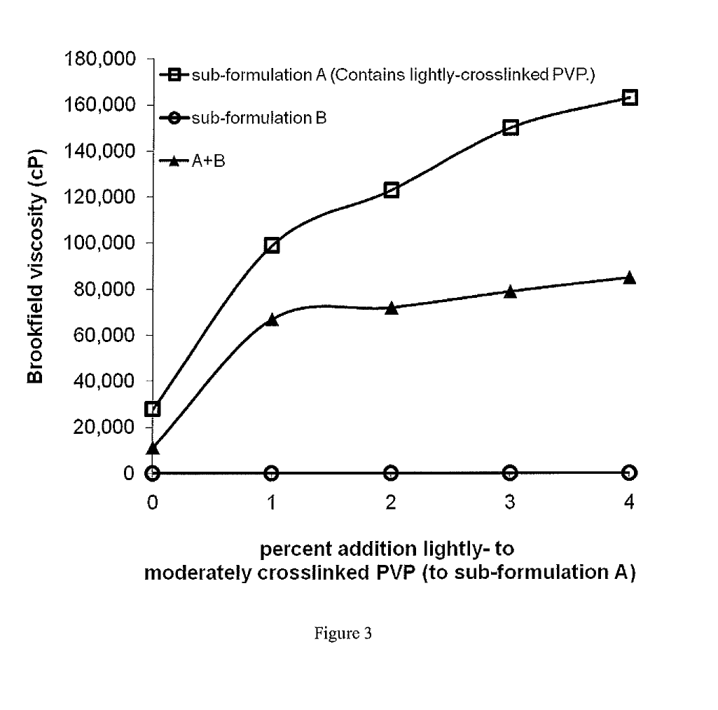

Hair relaxer formulations of Example 4 exhibited remarkably higher viscosities (FIG. 3) than the control. Hair relaxers containing 5% and 6% lightly-to moderately-crosslinked PVP were too thick to measure with the Brookfield viscometer.

### Example 6

### pH of Hair Relaxers of Example 4

The pH was measured for sub-formulation A, sub-formulation B, and the blended, final products of Example 4.

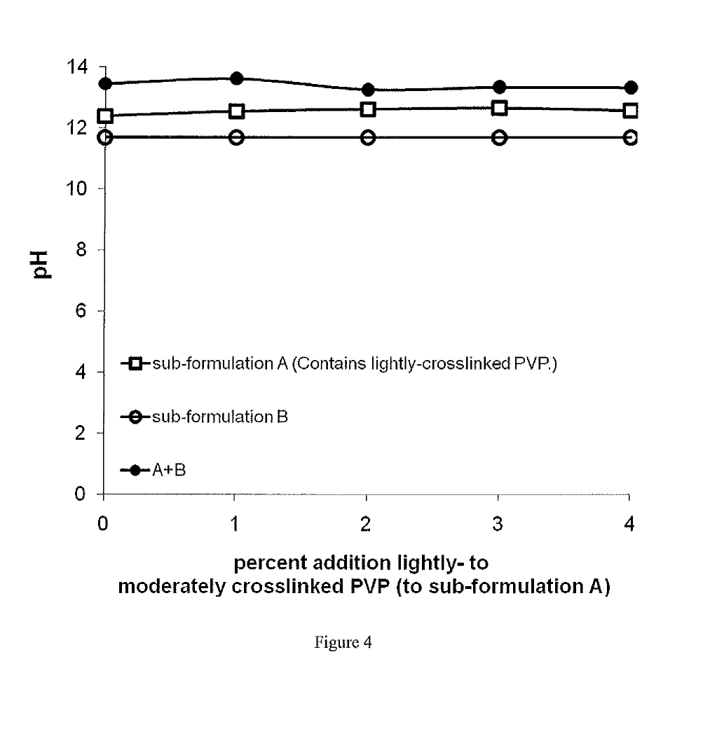

The increase in viscosity reported in Example 5 was not due to a drop in mixture pH, as all components, including the mixture, had a pH greater than 11.75 (FIG. 4).

### Example 7

### Viscosity and pH Stability of Hair Relaxers of Example 4

The control and six formulations of the invention from Example 4 were stored under four conditions: room temperature (about 22° C.), 40° C., 50° C., -20° C., sunlight window exposure, and a freeze/thaw cycle. After 1, 2, and 3 months storage samples were withdrawn and the viscosity and pH measured.

The crème relaxer (i.e., Sub-formulation A) of Example 4 maintained stable viscosity and pH for all stability storage conditions.

### Example 8

### Hair Relaxer Formulations with Lightly-to Moderately-Crosslinked PVP in the Activator (Sub-Formulation B).

Six hair relaxer formulations of the invention were prepared in two sub-formulations that yielded guanidine hydroxide when mixed (Table 6). Sub-formulation A was a cream relaxer and contained an oil-based phase 1 and a water-based phase 2. Sub-formulation B contained the activator, guanidine carbonate, and a total of 1% to 6% lightly-to moderately-crosslinked PVP, supplied from a 10% solution in water.

TABLE 6
Hair relaxer formulations of Example 8

| | |
|---|---|
| ingredient | mass percent |

Sub-formulation A: Crème Relaxer

| | Phase 1 | | | | | |
|---|---|---|---|---|---|---|
| petrolatum | 16 | 16 | 16 | 16 | 16 | 16 |
| mineral oil | 16 | 16 | 16 | 16 | 16 | 16 |
| cetearyl alcohol | 6 | 6 | 6 | 6 | 6 | 6 |
| Polysorbate-60 | 2 | 2 | 2 | 2 | 2 | 2 |
| PEG-75 lanolin | 2 | 2 | 2 | 2 | 2 | 2 |
| | Phase 2 | | | | | |
| deionized water | 51 | 51 | 51 | 51 | 51 | 51 |
| propylene glycol | 2 | 2 | 2 | 2 | 2 | 2 |
| calcium hydroxide | 5 | 5 | 5 | 5 | 5 | 5 |
| total | 100 | 100 | 100 | 100 | 100 | 100 |

Sub-formulation B: Activator

| | | | | | | |
|---|---|---|---|---|---|---|
| water | 65 | 55 | 45 | 35 | 25 | 15 |
| guanidine carbonate | 25 | 25 | 25 | 25 | 25 | 25 |
| lightly- to moderately | 10 | 20 | 30 | 40 | 50 | 60 |
| crosslinked PVP, | | | | | | |
| 10% solution | | | | | | |
| total | 100 | 100 | 100 | 100 | 100 | 100 |

### Example 9

### Viscosities of Hair Relaxers of Example 8

Viscosities were measured for sub-formulations A, sub-formulations B, and the combined mixtures of Example 8, A Brookfield RV viscometer fitted with spindle 4-7 (as appropriate) was employed for the viscosity measurements.

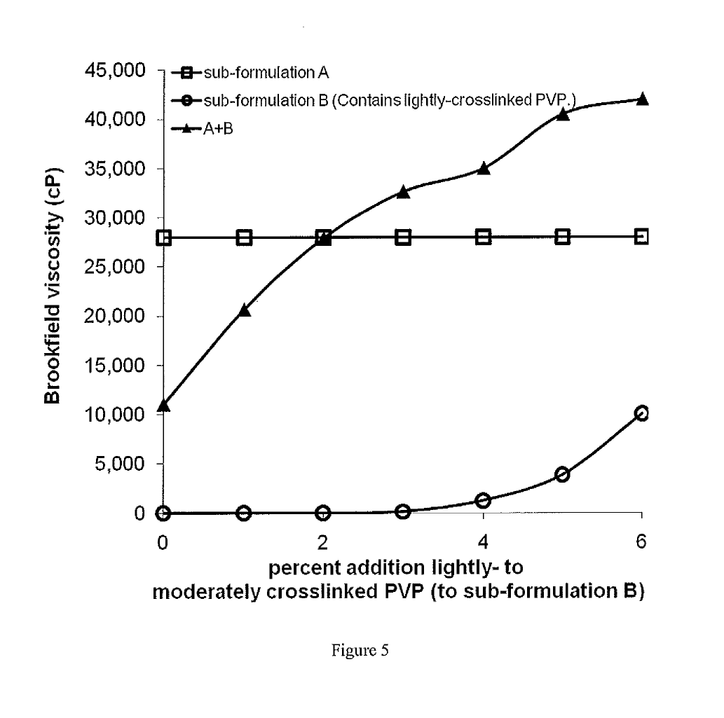

Formulations of the invention regained the original viscosity of the Creme Relaxer (sub-formulation A) with 2% addition of lightly-to moderately-crosslinked PVP (FIG. 5). At a viscosity of about 25,000 cP the hair relaxer presents a manageable consistency of a cream of limited dripping or running. Adding more than 2% lightly-to moderately-crosslinked PVP lead to higher Brookfield viscosities.

### Example 10

### pH of Hair Relaxers of Example 9

The pH was measured for sub-formulations A, sub-formulations B, and the blended, final products of Example 9.

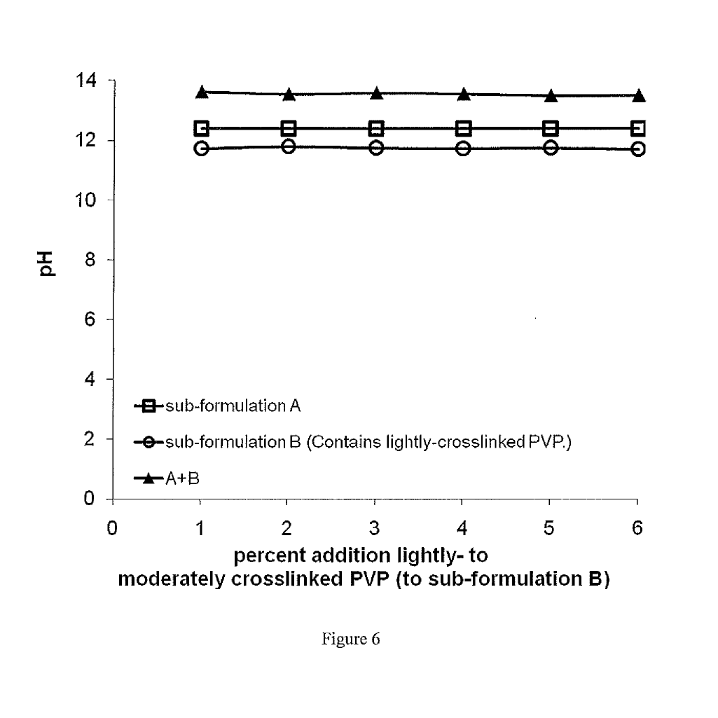

The increase in viscosity reported in Example 9 was not due to a drop in mixture pH, as all components, including the mixture, had a pH greater than 11.75 (FIG. 6).

### Example 11

### Viscosity and pH Stability of Liquid Activator (Sub-Formulation B) of Example 10

Viscosity and pH stability were tested for liquid activators (sub-formulation B) of Example 10 having 4%, 5%, and 6% lightly-crosslinked PVP. The samples were stored under six conditions: -20° C., 25° C., 40° C., 50° C., a −20° C/25° C. (freeze/thaw) cycle, and 25° C. with window exposure to natural sunlight. After I , 2, and 3 months storage samples were withdrawn and the viscosity and pH measured.

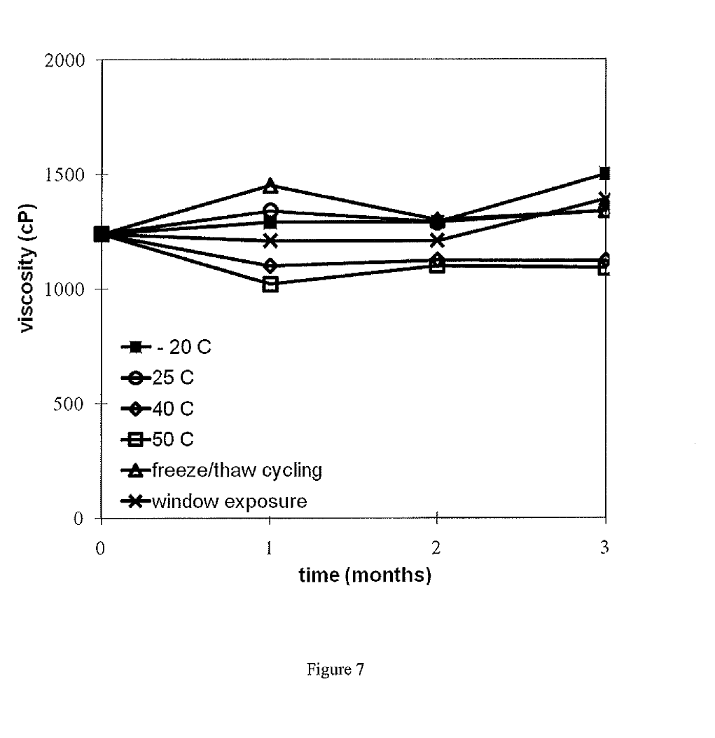

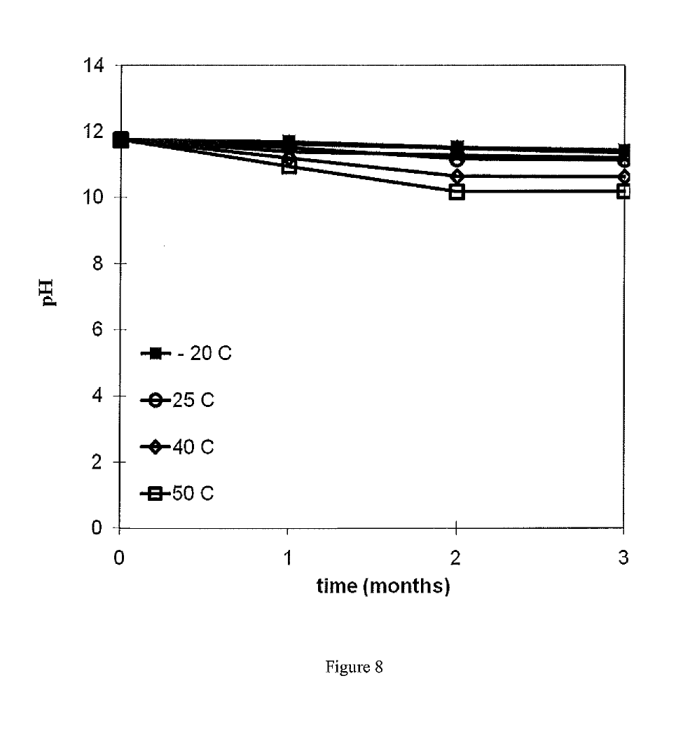

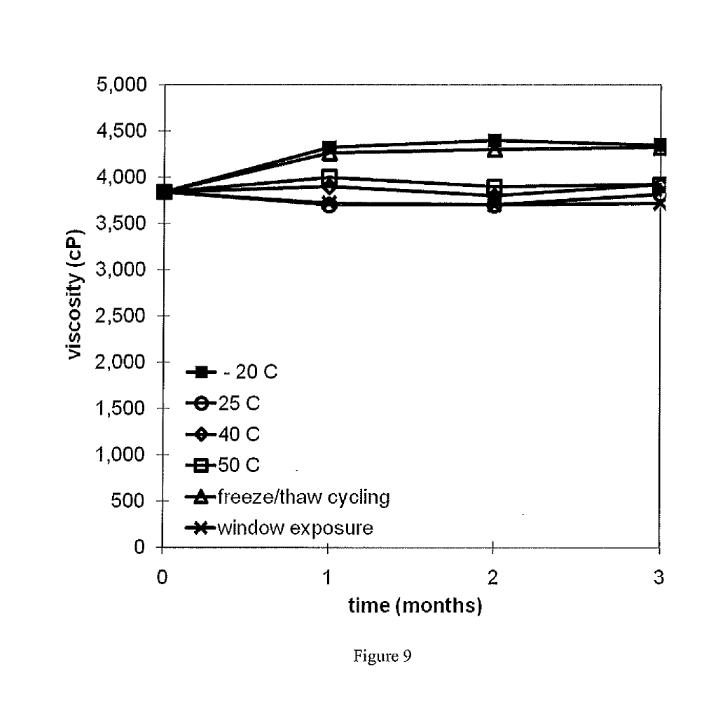

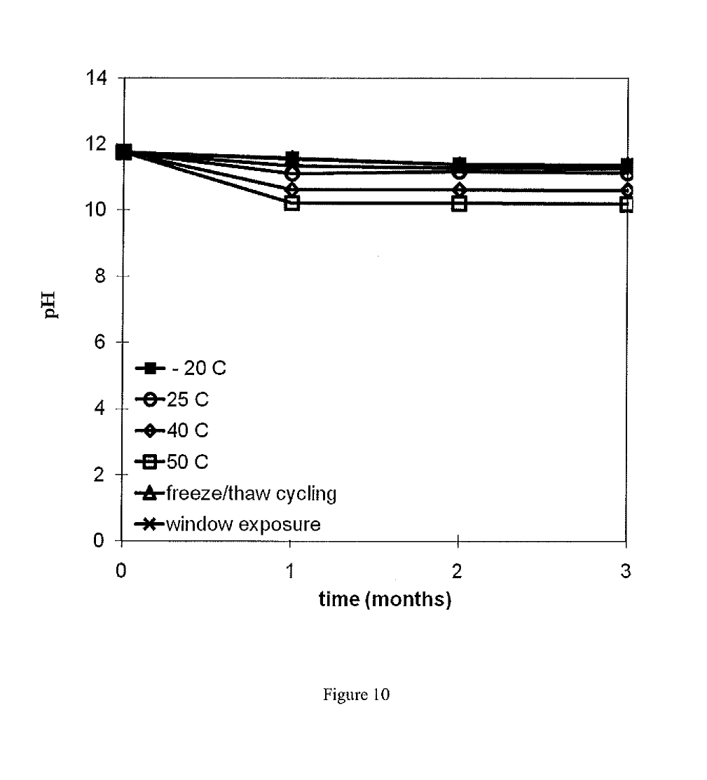

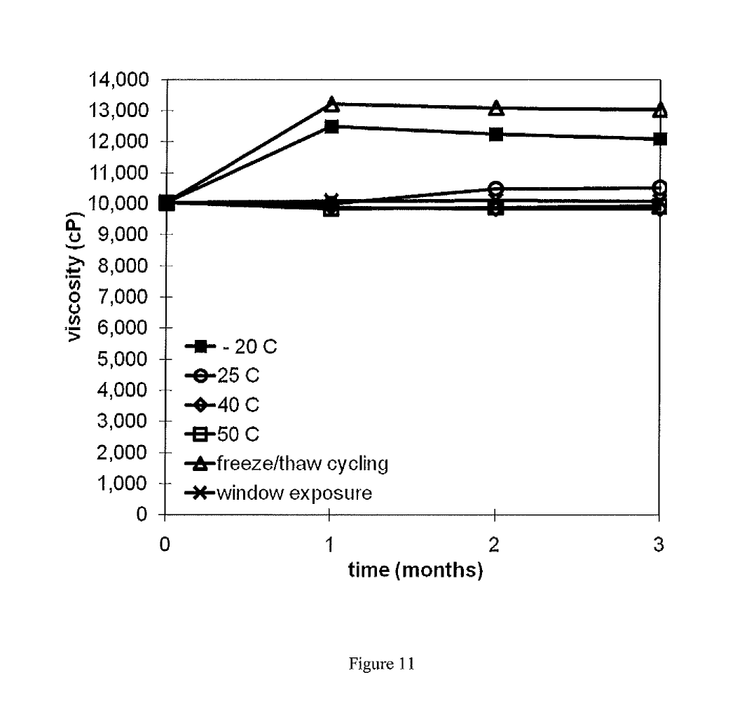

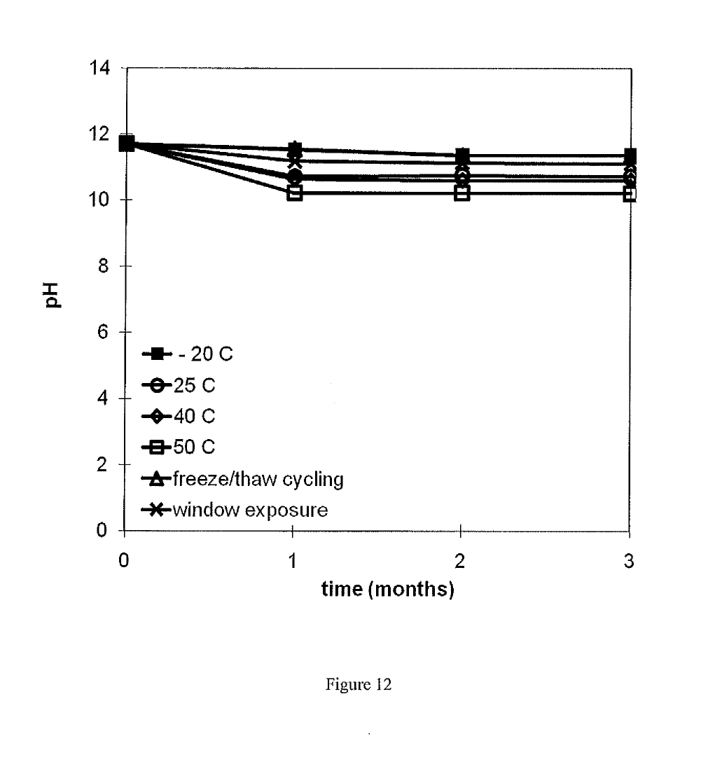

The liquid activators exhibited minimal changes in viscosity and pH over the three month test period that are representative of commercialized products (FIGS. 7-12).

### Example 12

### Performance of Thickened Hair Relaxer

The performance of the hair relaxer of Table 6 having 3% (w/w) lightly-to moderately-crosslinked PVP was compared to a commercially-available hair relaxer that contained about 95% of the same ingredients as the hair relaxer of the invention, but without any lightly-to moderately-crosslinked PVP. Each hair relaxer was applied to one Afro-textured hair tress, covering each hair strand with the relaxer from tress clamp to hair tip. The hair relaxers were left on for 20 minutes (per the instructions for the commercial product), and then the tresses were rinsed in clean water. The tresses were evaluated by a panel of experts with regard to hair shape, uniformity of shape, and shine.

Due in part to its rheological and sensory qualities, the hair relaxer having the lightly-to moderately-crosslinked PVP was easier to distribute on the hair tresses than the commercial product. The results suggested a favorable trend in shine: Four experts favored the shine of the hair relaxer of the invention, and two experts judged both products equal in shine. Overall hair shine is attributed, in part, to overall hair appearance, which includes hair alignment and uniformity.

The present invention has been described in detail with specific reference to particular embodiments thereof, but it will be understood that variations and modifications can be effected within the spirit and scope of the invention.

What is claimed is:

1. A composition comprising a mixture of at least two sub-formulations: (A) a first sub-formulation comprising a hydroxide, and (B) a second sub-formulation comprising an alkaline material, wherein either said first sub-formulation, or said second sub-formulation, or both said first and said second sub-formulations comprise an effective amount of lightly-to moderately-crosslinked poly(N-vinyl-2-pyrrolidone) (PVP).

2. The composition according to claim 1 wherein said first sub-formulation comprises lightly-to moderately-crosslinked PVP.

3. The composition according to claim 2 wherein said first sub-formulation comprises up to 10% (w/w) lightly-to moderately-crosslinked PVP, with respect to the total mass of said first sub-formulation.

4. The composition according to claim 3 having a viscosity of at least 500 cP.

5. The composition according to claim 2 having at least one oil-based phase.

6. The composition according to claim 5 wherein said oil-based phase comprises:
hydrocarbon oils, non-hydrocarbon oils, and blends thereof.

7. The composition according to claim 6 wherein said oil-based phase comprises a hydrocarbon oil selected from the group consisting of: petrolatum, mineral oil, vegetable oil, and blends thereof.

8. The composition according to claim 6 wherein said oil-based phase comprises a non-hydrocarbon oil comprising a silicon oil.

9. The composition according to claim 1 wherein said second sub-formulation comprises lightly-to moderately-crosslinked PVP.

10. The composition according to claim 9 wherein said second sub-formulation comprises up to 10% (w/w) lightly-to moderately-crosslinked PVP, with respect to the total mass of said second sub-formulation.

11. The composition according to claim 10 having a viscosity of at least 3,000 cP.

12. The composition according to claim 1 wherein said hydroxide is selected from the group consisting of calcium hydroxide, potassium hydroxide, lithium hydroxide, sodium hydroxide, ammonium hydroxide, and blends thereof.

13. The composition according to claim 1 wherein said alkaline material is selected from the group selected from: lithium carbonate, sodium carbonate, potassium carbonate, guanidine carbonate, glycolates, thioglycolates, and blends thereof.

14. The composition according to claim 1 having the form of a lotion, cream, paste, solution, or gel.

15. The composition according to claim 1 wherein said composition is a personal care composition.

16. The composition according to claim 15 wherein said personal care composition is a hair relaxer, a hair straightener, or a depilatory.

17. The composition according to claim 15 that further comprises an ingredient selected from the group consisting of: conditioners, fragrances, stabilizers, thickening agents, emulsifiers, buffering agents, moisturizers, and blends thereof.

18. The composition according to claim 1 wherein said composition is a performance chemicals composition.

19. The composition according to claim 18 wherein said performance chemicals composition is a degreaser, drain opener, paint stripper, cleaner, and leather depilator, or leather tanning composition.

20. A method of treating hair comprising the steps: (1) applying to the hair, skin, or scalp a composition comprising a mixture of at least two sub-formulations: (A) a first sub-formulation comprising a hydroxide, and (B) a second sub-formulation comprising an alkaline material, wherein at least one of said first sub-formulation, and said second sub-formulation, comprises an effective amount of lightly-to moderately-crosslinked PVP, (2) waiting between 1 and 60 minutes, and (3) rinsing said hair, skin, or scalp free of said composition.

21. The method according to claim 20 wherein said hair is at least: partially or fully straightened, partially or fully relaxed, fully or partially removed, curled, fully or partially permed, made less frizzy, made less bulky, made easier to comb, made easier to style, made softer, or made easier to style.

22. The method according to claim 20 wherein the composition has a viscosity of at least 500 cP.

23. The method according to claim 20 wherein said method provides at least: improved hair shine, better aligned hair, enhanced hair uniformity, or better looking hair compared to an equivalent method employing an equivalent product without lightly-to moderately-crosslinked PVP.

24. A system comprising:
(A) a first sub-formulation comprising a hydroxide; and
(B) a second sub-formulation comprising an alkaline material, wherein at least one of (A) and (B) comprises a lightly-to moderately-crosslinked poly(N-vinyl-2-pyrrolidone).

25. A system according to claim 24 wherein said system is a personal care system and a mixture of (A) and (B) provides a hair relaxer composition, a hair straightener composition or a depilator composition.
